(19) United States
(12) Patent Application Publication
(10) Pub. No.: US 2018/0209930 A1
(43) Pub. Date: Jul. 26, 2018

(54) DETECTION MODULE

(71) Applicant: CHANG GUNG UNIVERSITY, TAOYUAN CITY (TW)

(72) Inventors: SIDDHESWAR MAIKAP, TAOYUAN CITY (TW); SURAJIT JANA, TAOYUAN CITY (TW); JIAN-TAI QIU, NEW TAIPEI CITY (TW)

(21) Appl. No.: 15/410,860

(22) Filed: Jan. 20, 2017

(51) Int. Cl. G01N 27/22; H01L 29/16; G01N 33/487; G01N 27/406

(52) U.S. Cl. CPC G01N 27/227; G01N 27/406; G01N 33/487; H01L 29/16

(57) ABSTRACT

The present invention relates to a detection module. It uses a polycrystalline film underneath a porous sensing film to simultaneously contact with hydrogen peroxide having various concentrations, and the surface potential of materials of the polycrystalline film and the porous sensing film will be changed and resulting in voltage shift, which can be used to determine the concentration of hydrogen peroxide. Accordingly, the present invention can be applied to screen hydrogen peroxide related diseases as an auxiliary tool.

### FIELD OF THE INVENTION

The present invention relates generally to a detection module and particularly to the detection module capable of sensing hydrogen peroxide with higher sensitivity and can be used as lower detection limit.

### BACKGROUND OF THE INVENTION

Hydrogen peroxide (H₂O₂) is a kind of free radical, and it is a critical factor for mediating biological cycles. In addition, it has been used as a potential biomarker for oxidative stress diagnosis and a major catalyst for immune sensing according to recent researches.

In the prior arts, hydrogen peroxide detection relies on the use of the enzyme horse radish peroxidase (HRP) to oxidize its substrates and detects using a spectrophotometer. However, H₂O₂ sensing in a simple way with short time detection and with high specificity, is demanded for future disease diagnosis of the human body. For example, it is known that the content of the sarcosine in the urine or blood relates closely to the prostate cancer, and sarcosine react with H₂O to produce hydrogen peroxide in the urine or blood so that the higher concentration of the hydrogen peroxide the greater content of the sarcosine. In the purpose of screening the related disease more rapidly and more easily, enzyme-free electro-catalytic methods have gained the attention for H₂O₂ sensing.

In order to apply the hydrogen peroxide detection to the wilder range of disease diagnosis, the present invention provides a detection module that is used repeatedly and has higher sensitivity and detection efficiency. In addition, the detection method using the present invention is non-invasive, rapid and convenient.

### SUMMARY

An objective of the present invention is to provide a detection module includes a porous sensing film and a polycrystalline film disposed under the porous sensing film, and both materials of the porous sensing film and the polycrystalline film react with hydrogen peroxide.

Another objective of the present invention is to provide a detection module. Based on the principle that the surface potential of the materials of the porous sensing film and the polycrystalline film vary with hydrogen peroxide having different concentrations, the concentration of hydrogen peroxide can be determined by detecting the voltage change.

Still another objective of the present invention is to provide a detection module. Once a sample contains hydrogen peroxide, the voltage change owing to the surface potential change of the materials of the porous sensing film and the polycrystalline film can be detect and it further reflects the presence of biomarkers that produce hydrogen peroxide. Thereby, the present invention can be applied to screening a wild range of diseases like cancers.

A further objective of the present invention is to provide a detection module, which is succinct and can be popularized as commercial screening chips. In addition, the structure of the present invention can be used repeatedly. Thereby, screening for hydrogen peroxide related diseases can become convenient and the result can be given immediately.

Accordingly, the present invention discloses a detection module, which comprises a conductive substrate, a p-type silicon semiconductor layer, a silicon dioxide layer, a crystalline film, a porous sensing film, and a reference electrode. The p-type silicon semiconductor layer is disposed on the conductive substrate. The silicon dioxide layer is disposed on the p-type silicon semiconductor layer. The crystalline film is disposed on the silicon dioxide layer. The porous sensing film is disposed on the crystalline film for carrying a sample. The reference electrode is located above the porous sensing film for contacting the sample. Wherein a material of said crystalline film is selected from the group consisting of HfO₂, Ta₂O₅, Gd₂O₃, Al₂O₃, Cr₂O₃, WO₃, ZrO₂, MoOₓ, ErOₓ, YOₓ, PrOₓ, NbOₓ, ZnOₓ, LuOₓ, TmOₓ, HoOₓ, DyOₓ, YbOₓ, EuOₓ, TbOₓ, IGZOₓ, InNOₓ, NdOₓ, CeOₓ, NiO,ₓ GeOₓ, and SiOₓ.

### BRIEF DESCRIPTION OF THE DRAWINGS

FIG. 1 is shown a cross-sectional view of the detectionmodule according to the first embodiment of the present invention;

FIG. 2A is shown an enlarged partial cross-sectional view of an IrOₓ/Al₂O₃/SiO₂/p-Si structure according to the second embodiment of the present invention;

FIG. 2B is shown a high-resolution image of FIG. 2A;

FIG. 3A is shown an enlarged partial plane view of an IrOₓ partially coated over an Al₂O₃ layer in IrOₓ/Al₂O₃/SiO₂/p-Si structure according to the second embodiment of the present invention;

FIG. 3B is shown wild range view of FIG. 3A;

FIGS. 4A and 4B are shown binding energy test diagrams according to the second embodiment of the present invention;

FIG. 5A to 5D are shown pH sensitivity and linearity of TiOₓ, Al₂O₃, Ta₂O₅, and HfOₓ according to the third embodiment of the present invention;

FIG. 6A is shown a C-V characteristic diagram of an electrolyte/SiO₂/p-Si structure;

FIG. 6B is shown a C-V characteristic diagram of an electrolyte/IrOₓ/Ta₂O₅/SiO₂/p-Si structure according to the forth embodiment of the present invention;

FIG. 7A is shown a voltage test diagram of Gd₂O₃, HfO₂ and SiO₂; and

FIG. 7B is shown a voltage test diagram according to the fifth embodiment of the present invention.

### DETAILED DESCRIPTION

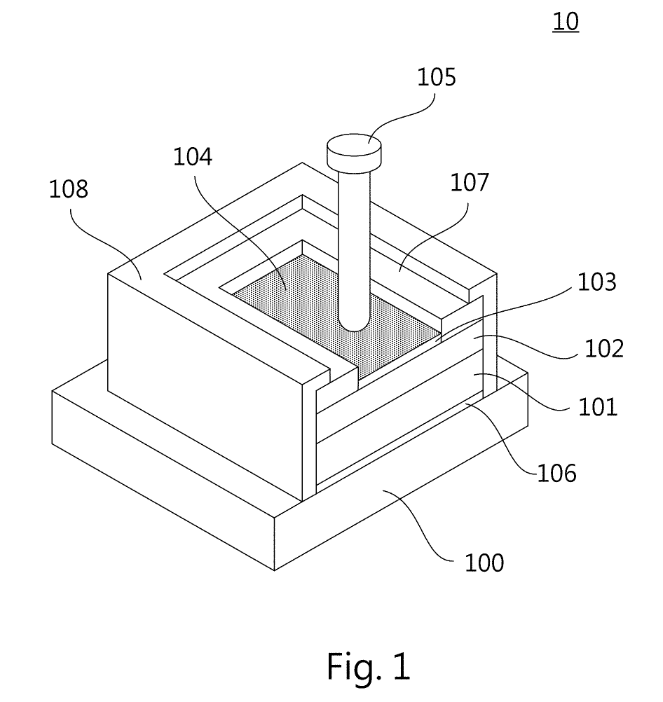

Please refer to FIG. 1, which shows a cross-sectional view of the detection module according to the first embodiment of the present invention. A detection module 10 comprises a conductive substrate 100, a p-type silicon semiconductor layer 101, a silicon dioxide layer 102, a polycrystalline film 103, a porous sensing film 104, and a reference electrode 105. The p-type silicon semiconductor layer 101 is disposed on the conductive substrate 100. The silicon dioxide layer 102 is disposed on the p-type silicon semiconductor layer 102. The polycrystalline film 103 is disposed on the silicon dioxide layer 102. The porous sensing film 104 is disposed on the polycrystalline film 103. The reference electrode 105 is located above the porous sensing film 104. Considering the ability in changing the surface potential of the material when contacts with hydrogen peroxide, the material of the polycrystalline film is selected from materials with the high-K value and it is preferably selected from the group consisting of HfO₂, Ta₂O₅, Gd₂O₃, Al₂O₃, Cr₂O₃, WO₃, ZrO₂, MoOₓ, ErOₓ, YOₓ, PrOₓ, NbOₓ, ZnOₓ, LuOₓ, TmOₓ, HoOₓ, DyOₓ, YbOₓ, EuOₓ, TbOₓ, IGZOₓ, InNOₓ, NdOₓ, CeOₓ, NiO,ₓ GeOₓ, and SiOₓ. Moreover, the material of the polycrystalline film is selected preferably from HfO₂ or Ta₂O₅.

In the structure of the detection module 10 according to the first embodiment, the conductive substrate 100 is a copper-plated printed circuit board and can be used as an electrode that corresponds to the reference electrode 105. The p-type silicon semiconductor layer 101 and the silicon dioxide layer 102 above the conductive substrate 100 achieve the characteristics of an electrolyte-insulator-semiconductor sensor. The fabrication methods for the p-type silicon semiconductor layer 101 and the silicon dioxide layer 102 are similar to normal semiconductor processes like chemical vapor deposition (CVD), plasma-enhanced CVD (PECVD), vapor deposition, e-gun vapor deposition, and radio-frequency (RF) sputtering, and layers composed of different materials can be stacked. In addition, an aluminum electrode layer 106 is further disposed between the p-type silicon semiconductor layer 101 and the conductive substrate 100 in order to increase the electrical conductivity. The material of the porous sensing film 104 is selected from the group consisting of IrOₓ, Pt, RuOₓ, Pd, Os, SnOₓ, MoS₂Oₓ, SmOₓ, and graphene oxide. The selection of the material is based on the ability in changing the surface potential when contacted with hydrogen peroxide and the porous sensing film 104 is lattice-matching to the polycrystalline film 103. Furthermore, because Si, Ge, Al, and Ti are easy to be oxidized in regular storage conditions, they are potential materials that used in the porous sensing film 104, however, an additional cleaning process is needed. Silver-silver chloride or other reference electrodes having a fixed potential difference can be selected to be the reference electrode 105.

The porous sensing film 104 made by IrOₓ does not cover completely the polycrystalline film 103 as a thick film. Instead, IrOₓ is distributed on the polycrystalline film 103 in the form of nanometer particles to form a nano-net type structure so that the materials of polycrystalline film 103 are partially exposed within the nano-net type structure and contact with the sample. The thickness of the porous sensing film is 2 to 10 nanometers and preferably 2 nanometers.

In addition, the polycrystalline film 103 is disposed below the porous sensing film 104 in order to increase the sensitivity and efficiency of the first embodiment. The materials can be preferably disposed on the silicon dioxide layer 102 by using sputtering and annealing at atemperature over 450° C. in ambient nitrogen to form polycrystalline grain (not shown in figures). The thickness of the polycrystalline film 103 is dependent on the polycrystalline grain size and preferably 5 to 20 nanometers.

Please refer to FIG. 1 again. When the first embodiment is operated, the liquid sample is dripped on the surface of the porous sensing film 104 and polycrystalline film 103. To prevent arbitrary flowing of the excess sample, one or more resin block(s) 107 may be further disposed on the silicon dioxide layer 102. These resin block(s) 107 partition and give a screening space above the silicon dioxide layer 102. The porous sensing film 104 and polycrystalline film 103 described above are located on the surface inside the screening space above the silicon dioxide layer 102. The material of the resin block(s) 107 can be SU-8, which is normally used as the negative photoresist. In the first embodiment, the material is baked and becomes the resin block(s) 107 for partitioning after spin coating.

In addition, the detection module 10 may further comprise a housing 108 formed by epoxy resin. The housing 108 protects the structure layers inside the detection module from the pollution or the oxidation and thus extending the lifetime of the detection module.

As described above, the detection module 10 disclosed in the first embodiment is operated as following, the reference electrode 105 is disposed near the porous sensing film 104 to give a first voltage, and then the sample is dripped on the surface of the porous sensing film 104 as well as the polycrystalline film 103 to give a second voltage. After that, comparing the first voltage and the second voltage for giving a difference value, and determine the content of hydrogen peroxide in the sample according to the difference value. The detection module 10 disclosed in the first embodiment is used to detect C-V (capacitance-to-voltage) changes following electrochemical reactions described below and hence determining whether the sample like urine, serum or blood sample contains hydrogen peroxide.

When IrOₓ of the porous sensing film 104 contact with hydrogen peroxide, the following reduction reaction occur:

Ir₂O₃+H₂O₂↔2IrO₂+H₂O (1)

In Formula (1), Ir₂O₃ react with hydrogen peroxide and then produce IrO₂. The Ir³⁺ oxidation state changes to Ir⁴⁺ state in contact of hydrogen peroxide and it leads voltage shifts. On the other hand, when GdOₓ of the polycrystalline film 103 contact hydrogen peroxide, the following reduction reactions occur:

Gd↔Gd²⁺+2e⁻↔Gd³⁺+3e⁻ (2)

H₂O₂+e⁻↔OH⁻+OH* (3)

OH*+e⁻↔OH⁻ (4)

2OH⁻+2H⁺↔2H₂O (5)

By following above equations (2) to (5), the oxidation state of Gd changes from Gd²⁺ to Gd³⁺. The H⁺ ions are supplied by buffer solutions. The voltage shift increases with increasing H₂O₂ concentration because the generation of Gd³⁺ ions increases.

Besides, while the detection module according to the first embodiment is operated, a buffer solution may be further added between the porous sensing film and the reference electrode. The function of this buffer solution is to influence the pH value of the sample and thus adjusting the substrate bias.

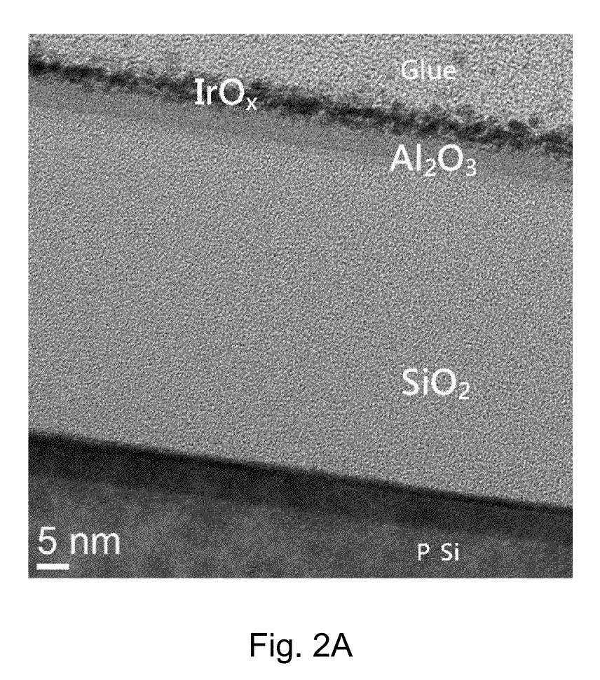

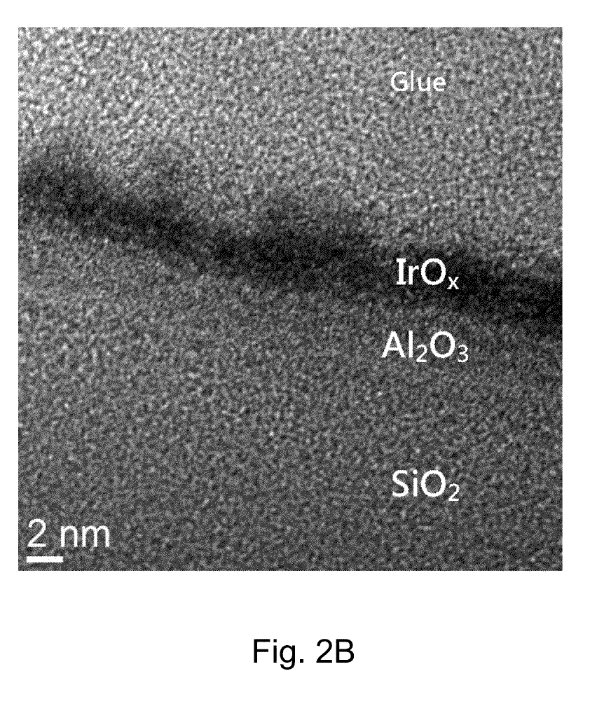

Please refer to FIGS. 2A and 2B, which show an enlarged partial cross-sectional view of an IrOₓ/Al₂O₃/SiO₂/p-Si structure according to the second embodiment of the present invention and a high-resolution image of FIG. 2A. The transmission electron microscope (TEM) images clearly show the IrOₓ/Al₂O₃/SiO₂/p-Si structure and the thickness of Al₂O₃ composed polycrystalline film is approximately 5 nm.

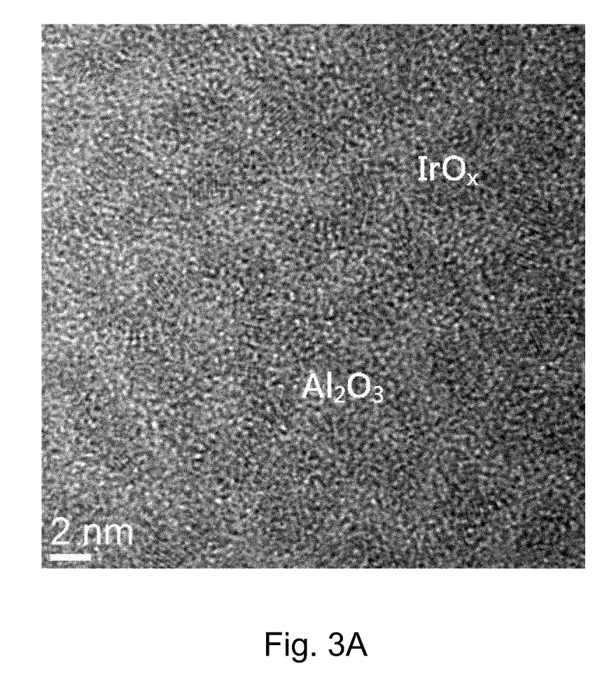

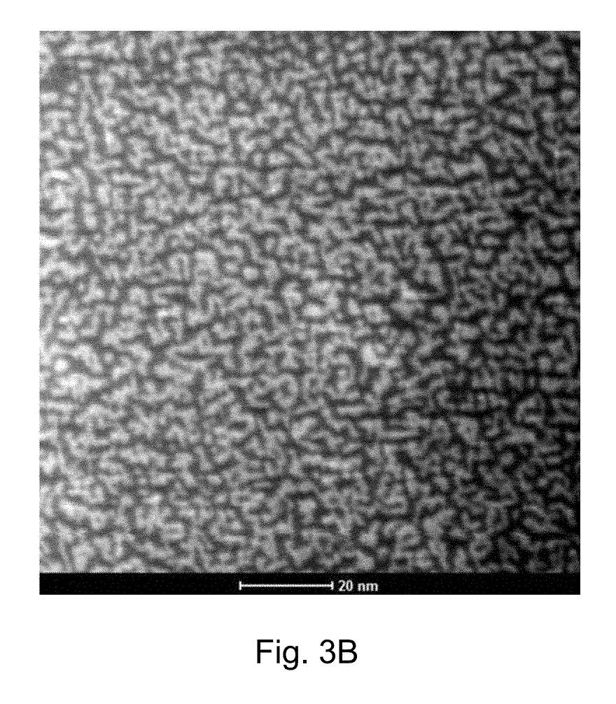

Please refer to FIGS. 3A and 3B, which show an enlarged partial plane view of an IrOₓ partially coated over an Al₂O₃ film in IrOₓ/Al₂O₃/SiO₂/p-Si structure according to the second embodiment of the present invention and a wild range view of FIG. 3A. As shown in FIG. 3A, IrOₓ shows crystalline while Al₂O₃ shows amorphous as marked. In addition, the IrOₓ shows nano-net type in FIG. 3B, while white wires are IrOₓ and black regions indicate Al₂O₃.

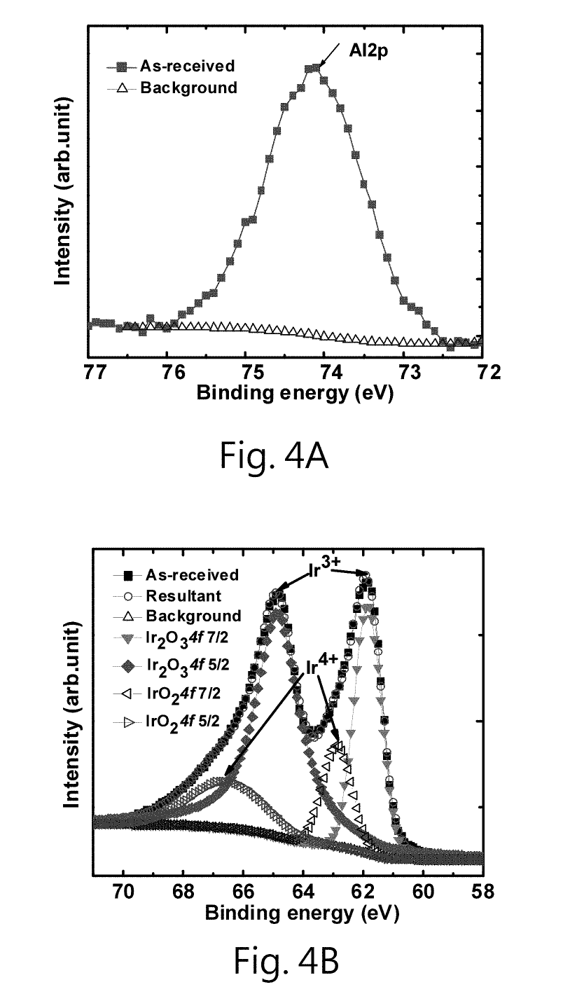

Please refer to FIGS. 4A and 4B, which show binding energy test diagrams according to the second embodiment of the present invention. As shown in figures, FIG. 4A shows X-ray photo-electron spectroscopy (XPS) data of the Al2p core-level electrons from the IrOₓ/Al₂O₃/SiO₂/p-Si structure and FIG. 4B shows X-ray photo-electron spectroscopy (XPS) data of the Ir4f core-level electrons from the IrOₓ/Al₂O₃/SiO₂/p-Si structure. The binding energy peak of Al2p electron is 74.13 eV, which corresponds to Al₂O₃composed polycrystalline film underneath IrOₓ composed porous sensing film. The 2 nm-thick IrOₓ composed porous sensing film is found to be consist of Ir³⁺ and Ir⁴⁺ oxidation states at oxygen content of 50% is shown. The characteristic peaks of Ir³⁺ (61.9 eV and 64.8 eV) and Ir⁴⁺ (62.8 eV and 66.6 eV) are observed. The reversible redox switching between these two states owing to partial hydroxylation in contact with water and sensitive towards hydroxyl ions, which are contributed to various hydrous forms of Ir³⁺/Ir⁴⁺ and Ir⁴⁺/Ir⁵⁺ redox couple, which will be useful for oxidation/reduction (redox) properties.

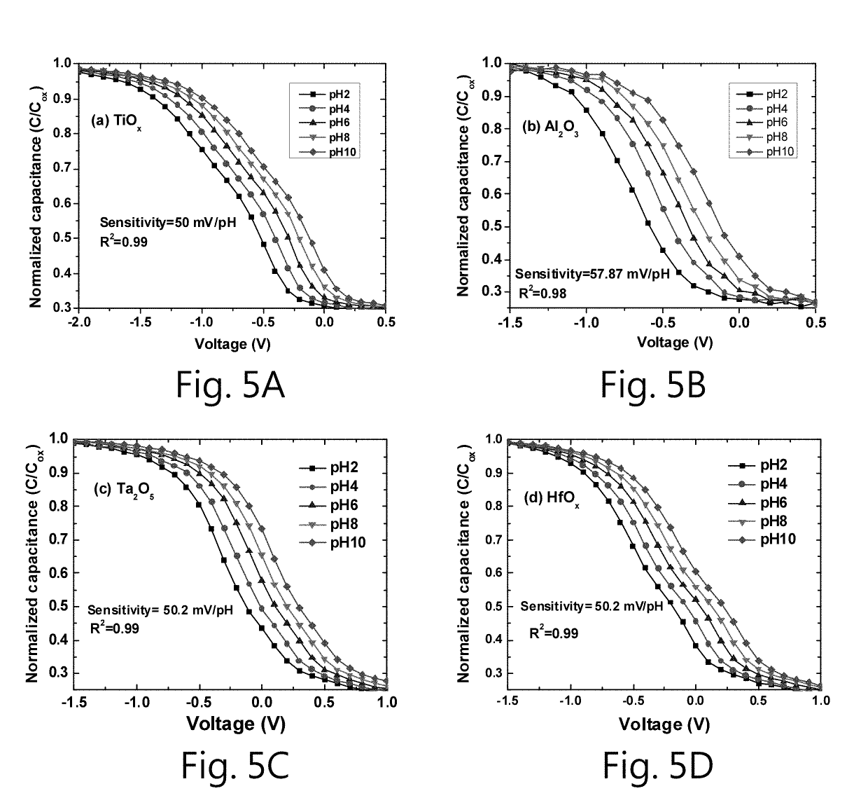

Please refer to FIG. 5A to 5D, which show pH sensitivity and linearity of TiOₓ, Al₂O₃, Ta₂O₅, and HfOₓ according to an embodiment of the present invention. The data show that these materials formed underneath of the porous sensing film to have high pH sensitivity and excellent catalytic effect for lower detection limit of hydrogen peroxide sensing.

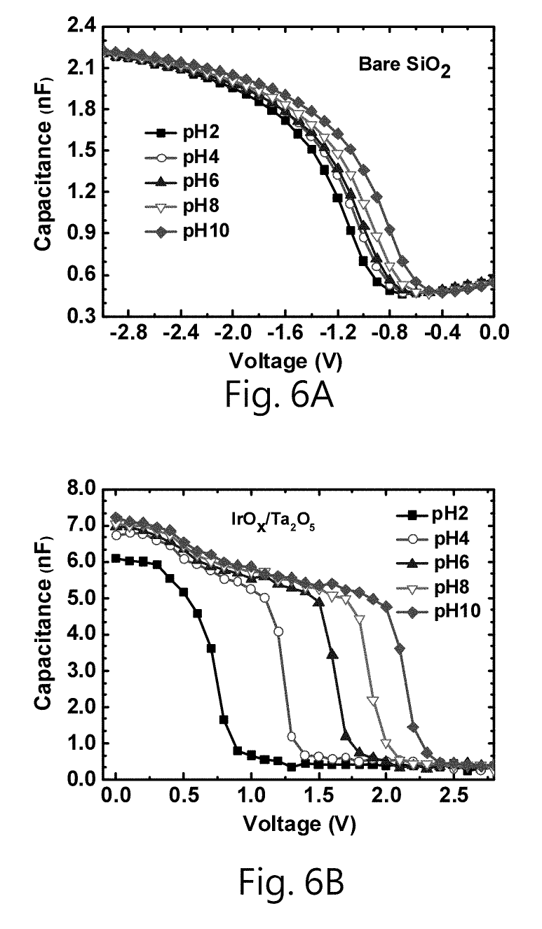

Please refer to FIGS. 6A and 6B, the figures show a C-V characteristic diagram of an electrolyte/SiO₂/p-Si structure and a C-V characteristic diagram of an electrolyte/IrOₓ/Ta₂O₅/SiO₂/p-Si structure according to the forth embodiment of the present invention. The accumulation capacitance of the electrolyte/IrOₓ/Ta₂O₅/SiO₂/p-Si structure is approximately 3 times higher than the electrolyte/SiO₂/p-Si structure (7nF in FIG. 6B compared with 2.2 nF in FIG. 6A), which is owing to the porous sensing film with porous nature of IrOₓ nano-net structure. Similarly, the highest sensitivity of the porous TrOₓ nano-structure is observed because of higher active binding sites, a higher work function from oxidation, and a higher dielectric permittivity than a bare SiO₂ layer.

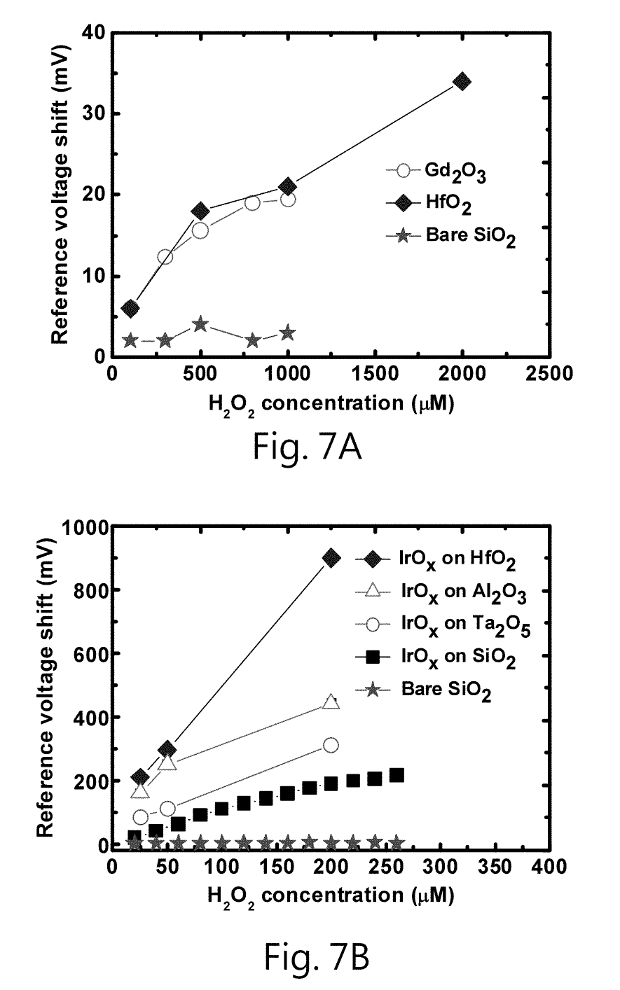

Please refer to FIGS. 7A and 7B, which show a voltage test diagramof Gd₂O₃, HfO₂ and SiO₂ and a voltage test diagram according to the fifth embodiment of the present invention. As shown in FIG. 7A, a good reference voltage (Vr) shifts are observed owing to different oxidation states, for example, Gd¹⁺, Gd²⁺, and Gd³⁺ oxidation states in a pH 7 buffer solution including H₂O₂. On the other hand, the bare SiO₂ layer does not show H₂O₂ sensing. Next, as shown in FIG. 7B, the surface site sensitivity of TrOₓ/HfO₂, TrOₓ/Al₂O₃, TrOₓ/Ta₂O₅ or TrOₓ/SiO₂ structures allow hydrogen peroxide detection in PBS buffer at pH 7.4. The structure including TrOₓ composed porous sensing film and underneath polycrystalline film displayed higher hydrogen peroxide sensing compare to structures in FIG. 7A due to redox active sites, although the structure including bare SiO₂ layer did not exhibit hydrogen peroxide sensing.

To sum up, the present invention discloses a detection modulein detail. The sample for detection is urine, serumor blood. If the urine, serum or blood contains hydrogen peroxide, the materials of a porous sensing film and a polycrystalline film react with the hydrogen peroxide and changing the surface potentialof the materials and leads voltage shifts. By detecting the voltage shifts, the concentration of the hydrogen peroxide can be determined. The present invention owns the feature of rapid screening and high sensitivityas an auxiliary tool for disease diagnosis.

Accordingly, the present invention conforms to the legal requirements owing to its novelty, non-obviousness, and utility. However, the foregoing description is only embodiments of the present invention, not used to limit the scope and range of the present invention. Those equivalent changes or modifications made according to the shape, structure, feature, or spirit described in the claims of the present invention are included in the appended claims of the present invention.

What is claimed is:

1. A detection module, comprising:
a conductive substrate;
a p-type silicon semiconductor layer disposed on said conductive substrate;
a silicon dioxide layer disposed on said p-type silicon semiconductor layer;
a polycrystalline film disposed on said silicon dioxide layer;
a porous sensing film disposed on said polycrystalline film for carrying a sample; and
a reference electrode located above said porous sensing film for contacting said sample;
wherein a material of said crystalline film is selected from the group consisting of HfO₂, Ta₂O₅, Al₂O₃, Gd₂O₃, Cr₂O₃, WO₃, ZrO₂, MoOₓ, ErOₓ, YO,, PrOₓ, NbOₓ, ZnOₓ, LuOₓ, TmOₓ, HoOₓ, DyOₓ, YbOₓ, EuOₓ, TbOₓ, IGZOₓ, InNOₓ, NdOₓ, CeOₓ, NiO,ₓ GeOₓ, and SiOₓ.

2. The detection module of claim 1, wherein said conductive substrate is a copper-plated printed circuit board.

3. The detection module of claim 1, wherein an aluminum electrode layer is disposed between said p-type silicon semiconductor layer and said conductive substrate.

4. The detection module of claim 1, wherein the material of said porous sensing film is selected from the group consisting of IrOₓ, Pt, RuOₓ, Pd, Os, SnOₓ, MoS₂Oₓ, SmOₓ, and graphene oxide.

5. The detection module of claim 1, and further comprising at least one resin block on said polycrystalline film for partitioning and giving a screening space, and said porous sensing film located on said polycrystalline film in said screening space.

6. The detection module of claim 1, wherein a thickness of said silicon dioxide layer is between 20 and 40 nanometers.

7. The detection module of claim 1, wherein the thickness of said crystalline film is between 5 and 20 nanometers.

8. The detection module of claim 1, wherein the thickness of said porous sensing film is between 2 and 10 nanometers.

9. The detection module of claim 1, wherein said sample is urine, serum or blood.
